(19) United States
(12) Patent Application Publication
(10) Pub. No.: US 2023/0128032 A1
(43) Pub. Date: Apr. 27, 2023

(54) Early Drug Interventions to Reduce COVID-19 Related Respiratory Distress, Need for Respirator Assist and Death

(71) Applicant: Curtails LLC, Smyrna, GA (US)

(72) Inventor: Mark G. Currie, Boston, MA (US)

(21) Appl. No.: 17/915,247

(22) PCT Filed: Mar. 30, 2021
(86) PCT No.: PCT/US2021/024909

§ 371 Date: Sep. 28, 2022

Related U.S. Application Data
(60) Provisional application No. 63/002,530, filed on Mar. 31, 2020.
(60) Provisional application No. 63/006,990, filed on Apr. 8, 2020.
(60) Provisional application No. 63/026,592, filed on May 18, 2020.
(60) Provisional application No. 63/054,927, filed on Jul. 22, 2020.
(60) Provisional application No. 63/160,057, filed on Mar. 12, 2021.

(51) Int. Cl. A61K 38/22; A61K 45/06; C07K 14/58

(52) U.S. Cl. CPC A61K 38/2242; A61K 45/06; C07K 14/58

(57) ABSTRACT

The present invention provides methods of treating the early stages of COVID-19 infection in a patient in need thereof. The methods comprise administering a therapeutically effective amount of a drug that increases signaling through the GC-A receptor, or a pharmaceutically acceptable salt thereof, to a patient in need thereof. In certain aspects, the drug is a NEP inhibitor, a GC-A receptor agonist, or a pharmaceutically acceptable salt thereof. In certain embodiments, the methods of the present invention prevent the progression from the early stages of COVID-19 infection to hypoxemia, acute respiratory distress syndrome (ARDS) or death. In certain embodiments, the methods of the present invention prevent the development of pulmonary dysfunction after developing early symptoms of COVID-19 infection or the progression from mild to severe hypoxemia in these patients.

### RELATED APPLICATIONS

This application claims the benefit of the filing date, under 35 U.S.C. § 119(e), of U.S. Provisional Application No. 63/002,530, filed on Mar. 31, 2020; U.S. Provisional Application No. 63/006,990, filed on Apr. 8, 2020; U.S. Provisional Application No. 63/026,592, filed on May 18, 2020; U.S. Provisional Application No. 63/054,927, filed on Jul. 22, 2020; and U.S. Provisional Application No. 63/160,057, filed on Mar. 12, 2021. The entire contents of each of the aforementioned applications are incorporated herein by reference.

### BACKGROUND

Coronavirus disease 2019 (COVID-19) is an infectious disease caused by severe acute respiratory syndrome with coronavirus 2 infection (SARS-CoV-2). The disease was first identified in 2019 in Wuhan, the capital of China's Hubei province, and has since spread globally, resulting in an ongoing 2019-20 coronavirus pandemic.

Common symptoms include fever, cough, and shortness of breath. Other symptoms may include muscle pain, sputum production, diarrhea, sore throat, loss of smell, and abdominal pain. While the majority of cases result in mild symptoms, others progress to pneumonia and multi-organ failure. As of Mar. 28, 2020, the overall rate of deaths per number of diagnosed cases was 4.6 percent; ranging from 0.2 percent to 15 percent according to age group, geographical location and existence of other concomitant health problems or comorbidities.

The virus is mainly spread during close contact and via respiratory droplets produced when people cough or sneeze. Respiratory droplets may be produced during breathing, but the virus is not generally airborne. People may also contract COVID-19 by touching a contaminated surface and then their face. It is most contagious when people are symptomatic, although spread may be possible before symptoms appear. The virus can live on surfaces up to 72 hours. Time from exposure to onset of symptoms is generally between two and fourteen days, with an average of five days. The standard method of diagnosis is by reverse transcription polymerase chain reaction (rRT-PCR) from a nasopharyngeal swab. The infection can also be diagnosed from a combination of symptoms, risk factors and a chest CT scan showing features of pneumonia.

Recommended measures to prevent infection include frequent hand washing, social distancing (maintaining physical distance from others, especially from those with symptoms), covering coughs and sneezes with a tissue or inner elbow, and keeping unwashed hands away from the face. The use of masks is recommended by some national health authorities for those who suspect they have the virus and their caregivers.

As of the time of filing of this application, there is no vaccine or specific antiviral treatment for COVID-19. Current stage of management involves treatment of symptoms, supportive care, isolation, and experimental measures, including the use of some experimental drug treatments, such as influenza or anti-malarial medication. There are currently no approved treatments for the treatment of COVID-19 complications.

There is an acute need for early drug interventions that prevent progression of COVID-19 infection to pulmonary dysfunction (including pulmonary edema and inflammation), hypoxemia, acute respiratory distress syndrome (ARDS) and, eventually to death. More specifically, there is a need for therapeutic interventions that preserve endothelial barrier function in the lung and protect the lung from injury.

Furthermore, several studies have reported a high prevalence of cardiovascular disease including hypertension, obesity and diabetes in hospitalized patients with COVID-19. In addition, it has been observed that underlying cardiovascular (CV) disease, such as hypertension, obesity, and diabetes, is associated with a higher risk of progression to severe hypoxemia, ARDS, and death in COVID-19-infected patients. The presence of hypertension has emerged asone of such underlying CV risk factors. Hypertension is a complex disease with many different contributing genes and environmental factors. As the COVID-19 pandemia has been evolving and more data gathered, it has been recognized that in the US, older individuals and African Americans are two populations that appear to have a higher degree of progression of COVID-19 infection to hypoxemia, ARDS and death. It is also known that older individuals and African Americans have a higher degree of salt sensitivity and develop salt-sensitive hypertension at a higher rate than other populations. African Americans in the US, in particular, are known to have age-adjusted prevalence of hypertension of 44.4% and 43.9% in black men and women, respectively, vs 34.1% and 30.3% in non-Hispanic white men and women.

It is our hypothesis that the pathobiology underlying hypertension and salt sensitivity in these populations may also be the reason for their increased vulnerability to COVID-19-induced ARDS and other complications, including but not limited to, lung complications, kidney injury, heart complications and coagulopathies. One biological key factor that has been associated with salt sensitivity is impaired secretion of atrial natriuretic peptide (ANP), which leads to reduced signaling through the guanylate cyclase-A (GC-A) receptor-cGMP pathway. Natriuretic peptides are key hormones in the regulation of vascular tone and kidney function, and there is clear evidence that impairment of this system contributes to hypertension, including but not limited to, salt-sensitive hypertension. Obesity, another COVID-19 risk factor, has also been associated with lower circulating natriuretic peptide levels. There is also evidence that a natriuretic peptide treatment approach has the potential to attenuate the coagulopathy associated with COVID-19. The cardioprotective effects of natriuretic peptides (Wang, D., Gladysheva, I. P., Fan, T. H., Sullivan, R., Houng, A. K., and Reed, G. L. (2014) Atrial natriuretic peptide affects cardiac remodeling, function, heart failure, and survival in a mouse model of dilated cardiomyopathy. Hypertension 63, 514-519) may also attenuate COVID-19 myocarditis and acute cardiovascular syndrome.

### SUMMARY

New drug treatments that prevent progression of COVID-19 to ARDS and associated complications (including hypoxemia, and death) by protecting the lung endothelial barrier and reducing lung hyperpermeability are one object of this invention.

New early drug interventions that protect patient populations at high risk of developing complications after COVID-19 infection, by restoring signaling through the (ANP-BNP)-GC-A receptor pathway to normal levels and preventing progression to pulmonary dysfunction, including pulmonary inflammation and pulmonary edema, and subsequent associated complications, namely hypoxemia, ARDS and death is another object of this invention. In some embodiments, these treatments are also protective for the kidney. In other embodiments, these treatments may also protect the patient from the development of coagulopathies. In still other embodiments, these treatments may also protect the patient from the development of heart complications, such as myocarditis and acute cardiovascular syndrome.

In one aspect, the invention provides a method of treating the early stages of COVID-19 infection, comprising administering a therapeutically effective amount of a drug that increases signaling through the guanylate cyclase-A (GC-A) receptor, or a pharmaceutically acceptable salt thereof, to a patient in need thereof.

In another aspect, the invention provides a method of preventing the progression from the early stages of COVID-19 infection to hypoxemia, ARDS or death, comprising administering a therapeutically effective amount of a drug that increases signaling through the GC-A receptor, or a pharmaceutically acceptable salt thereof, to a patient in need thereof.

In another aspect, the invention provides a method of preventing the development of pulmonary dysfunction after developing early symptoms of COVID-19 infection, comprising administering a therapeutically effective amount of a drug that increases signaling through the GC-A receptor, or a pharmaceutically acceptable salt thereof, to a patient in need thereof. In some embodiments, pulmonary dysfunction is associated with or manifested as pulmonary edema. In other embodiments, pulmonary dysfunction is associated with or manifested as pulmonary inflammation.

In another aspect, the invention provides a method of preventing the progression to severe hypoxemia from mild hypoxemia in a patient with COVID-19, comprising administering a therapeutically effective amount of a drug that increases signaling through the guanylate cyclase-A (GC-A) receptor, or a pharmaceutically acceptable salt thereof, to a patient in need thereof.

In one aspect, the invention provides a method of treating the early stages of COVID-19 infection, comprising administering a therapeutically effective amount of a neural endopeptidase (NEP) inhibitor, or a pharmaceutically acceptable salt thereof to a patient in need thereof.

In another aspect, the invention provides a method of preventing the progression from the early stages of COVID-19 infection to hypoxemia, ARDS or death, comprising administering a therapeutically effective amount of a NEP inhibitor, or a pharmaceutically acceptable salt thereof to a patient in need thereof.

In another aspect, the invention provides a method of preventing the development of pulmonary dysfunction after developing early symptoms of COVID-19 infection, comprising administering a therapeutically effective amount of a NEP inhibitor, or a pharmaceutically acceptable salt thereof to a patient in need thereof. In some embodiments, pulmonary dysfunction is associated with or manifested as pulmonary edema. In other embodiments, pulmonary dysfunction is associated with or manifested as pulmonary inflammation.

In another aspect, the invention provides a method of preventing the progression to severe hypoxemia from mild hypoxemia in a patient with COVID-19, comprising administering a therapeutically effective amount of a NEP inhibitor, or a pharmaceutically acceptable salt thereof to a patient in need thereof.

In another aspect, the invention provides a method of treating the early stages of COVID-19 infection, comprising administering a therapeutically effective amount of a GC-A receptor agonist or a pharmaceutically acceptable salt thereof to a patient in need thereof.

In another aspect, the invention provides a method of preventing the progression from the early stages of COVID-19 infection to hypoxemia, ARDS or death, comprising administering a therapeutically effective amount of a GC-A receptor agonist or a pharmaceutically acceptable salt thereof to a patient in need thereof.

In another aspect, the invention provides a method of preventing the development of pulmonary dysfunction after developing early symptoms of COVID-19 infection, comprising administering a therapeutically effective amount of a GC-A receptor agonist or a pharmaceutically acceptable salt thereof to a patient in need thereof. In some embodiments, pulmonary dysfunction is associated with or manifested as pulmonary edema. In other embodiments, pulmonary dysfunction is associated with or manifested as pulmonary inflammation.

In another aspect, the invention provides a method of preventing the progression to severe hypoxemia from mild hypoxemia in a patient with COVID-19, comprising administering a therapeutically effective amount of a GC-A receptor agonist or a pharmaceutically acceptable salt thereof to a patient in need thereof.

In another aspect, the invention provides pharmaceutical compositions comprising a NEP inhibitor, a GC-A agonist or a pharmaceutically acceptable salt thereof, for use in method for treating the early stages of COVID-19 infection, preventing the progression from the early stages of COVID-19 infection to hypoxemia, ARDS or death, preventing the development of pulmonary dysfunction after developing early symptoms of COVID-19 infection or preventing the progression from mild hypoxemia to severe hypoxemia in a patient in need thereof. Also included is the use of a NEP inhibitor, a GC-A agonist or a pharmaceutically acceptable salt thereof, for the manufacture of a medicament for treating the early stages of COVID-19 infection, preventing the progression from the early stages of COVID-19 infection to hypoxemia, ARDS or death, preventing the development of pulmonary dysfunction or preventing the progression from mild hypoxemia to severe hypoxemia after developing early symptoms of COVID-19 infection in a patient in need thereof. In some embodiments of the above pharmaceutical compositions, pulmonary dysfunction is associated with or manifested as pulmonary edema. In other embodiments, pulmonary dysfunction is associated with or manifested as pulmonary inflammation.

In some embodiments of the above methods, uses and pharmaceutical compositions, the treatments are also protective to the kidney from injury. In other embodiments, the treatments protect the patient in need thereof from the development of coagulopathies. In still other embodiments, these treatment may also protect the patient from the development of heart complications, such as myocarditis and acute cardiovascular syndrome.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls. The compounds described herein may be defined by their chemical structures and/or chemical names. Where a compound is referred to by both a chemical structure and a chemical name, and the chemical structure and chemical name conflict, the chemical structure is determinative of the compound's identity.

Drug Therapies and Biological Rationale

As of the time of filing of this application, there is no vaccine or specific antiviral treatment for COVID-19. Current stage of management involves treatment of symptoms, supportive care, isolation, and experimental measures, including the use of some experimental drug treatments, such as influenza or anti-malarial medication and others. There are currently no approved treatments for the treatment of COVID-19 complications, including hypoxemia and ARDS.

There is an acute need for early drug interventions that prevent progression of COVID-19 infection to pulmonary dysfunction (including pulmonary edema and inflammation), hypoxemia, ARDS and, eventually to death. These drugs will also reduce need for the utilization of ventilators in the hospital setting.

Therapeutic interventions that preserve endothelial barrier function in the lung and protect the lung from injury may be useful in preventing progression of COVID-19 infection to ARDS. Therefore, there is a need for therapeutic interventions that preserve endothelial barrier function in the lung and protect the lung from injury.

New drug treatments that prevent progression of COVID-19 to ARDS and associated complications by protecting the lung endothelial barrier and reducing lung hyperpermeability are one object of this invention.

Several animal and clinical studies have shown that pharmacological or genetic interventions that increase signaling through the natriuretic peptide receptor 1 (NPR1, also known as guanylate cyclase-A (GC-A) receptor)-cyclic GMP pathway prevent all drivers of increased vascular permeability in the lung and protect the lung from various types of injury. The importance of targeting the endothelium in COVID-19 patients is underscored by the fact that lungs from COVID-19 patients exhibit severe vascular endothelial injury (Ackermann, M., Verleden, S. E., Kuehnel, M., Haverich, A., Welte, T., Laenger, F., Vanstapel, A., Werlein, C., Stark, H., Tzankov, A., Li, W. W., Li, V. W., Mentzer, S. J., and Jonigk, D. (2020) Pulmonary Vascular Endothelialitis, Thrombosis, and Angiogenesis in Covid-19. N Engl J Med).

Thus, it is our hypothesis that therapeutic interventions targeting the GC-A-cyclic GMP pathway will prevent progression from the early stages of COVID-19 infection to hypoxemia, ARDS or death, by protecting the lung endothelium and reducing lung hyperpermeability.

In addition, it has been observed that underlying CV disease is associated with a higher risk of progression to severe hypoxemia, ARDS, and death in COVID-19-infected patients. As the COVID-19 pandemia has been evolving and more data gathered, it has also been recognized that older individuals and African Americans are two populations that appear to have a higher degree of progression of COVID-19 infection to hypoxemia, ARDS and death. It is also known that older individuals and African Americans have a higher degree of salt sensitivity and develop salt-sensitive hypertension at a higher rate than other populations. In the US, African Americans in particular, are known to have age-adjusted prevalence of hypertension of 44.4% and 43.9% in black men and women, respectively, vs 34.1% and 30.3% in non-Hispanic white men and women.

It is our hypothesis that the pathobiology underlying hypertension and salt sensitivity in these populations may also be the reason for their increased vulnerability to COVID-19-induced ARDS and other complications, including but not limited, to lung complications, kidney damage, heart complications as well as coagulopathies. One biological key factor that has been associated with salt sensitivity is impaired secretion of atrial natriuretic peptides, which leads to reduced signaling through the guanylate cyclase-A (GC-A) receptor-cGMP pathway. Natriuretic peptides are key hormones in the regulation of vascular tone and kidney function, and there is clear evidence that impairment of this system contributes to hypertension, including but not limited to, salt-sensitive hypertension. Obesity, another COVID-19 risk factor, has also been associated with lower circulating natriuretic peptide levels.

It is plausible that COVID-19-infected patients who already have deficiencies in the lung-protective natriuretic peptide hormone system are especially vulnerable to development of severe lung complications when infected with COVID-19.

The natriuretic peptides ANP and brain natriured peptide (BNP) are best known for their roles in the cardiovascular system. They were first identified as peptide hormones secreted by the heart. As agonists of the GC-A receptor, both ANP and BNP increase cyclic guanosine 3′5′-monophosphate (cGMP); and their pharmacologic effects on the vasculature (vasodilation) and kidney (natriuresis) have been well-documented. Furthermore, BNP and its precursor NT-proBNP are routine clinical laboratory measures used as prognostic markers in cardiology. NT-proBNP has been shown to be elevated in COVID-19 patients with severe disease and non-survivors, most likely an indication of increased cardiac stress (Gao L, Jiang D, Wen X S, Cheng X C, Sun M, He B, et al. Prognostic value of NT-proBNP in patients with severe COVID-19. Respir Res. 2020; 21(1):83; Han H, Xie L, Liu R, Yang J, Liu F, Wu K, et al. Analysis of heart injury laboratory parameters in 273 COVID-19 patients in one hospital in Wuhan, China. J Med Virol. 2020; Li J W, Han T W, Woodward M, Anderson C S, Zhou H, Chen Y D, et al. The impact of 2019 novel coronavirus on heart injury: A Systematic review and Meta-analysis. Prog Cardiovasc Dis. 2020).

Plasma ANP levels, which increase in normotensive subjects fed a high-salt diet, paradoxically decrease in black hypertensive subjects in response to a high-salt diet (Campese V M, Tawadrous M, Bigazzi R, Bianchi S, Mann A S, Oparil S, et al. Salt intake and plasma atrial natriuretic peptide and nitric oxide in hypertension. Hypertension. 1996; 28(3):335-40). In the Dallas Heart Study of 3148 individuals free of prevalent CV disease, hypertension was present in 41%, 25%, and 16% of black, white, and Hispanic individuals, respectively, while unadjusted NT-proBNP levels were lowest in black individuals (median: 24 pg/ml; interquartile range [IQR]: 10 to 52 pg/ml) as compared with white (32 pg/ml; IQR: 16 to 62 pg/ml), p<0.0001) and Hispanic individuals (30 pg/ml; IQR: 14 to 59 pg/ml) and white individuals. Similarly, levels of NT-proBNP, a precursor to BNP, were significantly lower in African Americans (43 pg/mL; IQR: 18 to 88 pg/ml) than Caucasians (68 pg/mL; IQR: 36 to 124 pg/ml; P<0.0001) in a large cohort study (Gupta DK, Claggett B, Wells Q, Cheng S, Li M, Maruthur N, et al. Racial differences in circulating natriuretic peptide levels: the atherosclerosis risk in communities study. J Am Heart Assoc. 2015; 4(5).)

This striking relationship between COVID-19 disease severity and natriuretic peptide levels can also be found in obese individuals. It is well-established from epidemiological studies that circulating natriuretic peptide levels are lower in obese individuals (Dessi-Fulgheri P, Sarzani R, Tamburrini P, et al. Plasma atrial natriuretic peptide and natriuretic peptide receptor gene expression in adipose tissue of normotensive and hypertensive obese patients. J Hypertens 1997; 15(12 Pt 2): 1695-9; Wang T J, Larson M G, Levy D, et al. Impact of obesity on plasma natriuretic peptide levels. Circulation 2004; 109(5): 594-600; Das S R, Drazner M R, Dries D L, et al. Impact of body mass and body composition on circulating levels of natriuretic peptides: results from the Dallas Heart Study. Circulation 2005; 112(14): 2163-8; Arora P, Reingold J, Baggish A, et al. Weight loss, saline loading, and the natriuretic peptide system. J Am Heart Assoc 2015; 4(1): e001265). Several studies have found that obese individuals are more likely to develop severe complications of COVID-19 (Cai Q, Chen F, Wang T, et al. Obesity and COVID-19 Severity in a Designated Hospital in Shenzhen, China. Diabetes Care 2020; Gao F, Zheng K I, Wang X B, et al. Obesity Is a Risk Factor for Greater COVID-19 Severity. Diabetes Care 2020; Kass D A, Duggal P, Cingolani O. Obesity could shift severe COVID-19 disease to younger ages. Lancet 2020; 395(10236): 1544-5).

There is also evidence that a natriuretic peptide treatment approach has the potential to attenuate the coagulopathy associated with COVID-19 (Zhou F, Yu T, Du R, et al. Clinical course and risk factors for mortality of adult inpatients with COVID-19 in Wuhan, China: a retrospective cohort study. Lancet 2020; 395 (10229): 1054-62; Yoshizumi M, Tsuji H, Nishimura H, et al. Natriuretic peptides regulate the expression of tissue factor and PAI-1 in endothelial cells. Thromb Haemost 1999; 82(5): 1497-503).

These observations in hight risk populations further support the use of agents that upregulate the CG-A pathway for the prevention of progression of early COVID-19 infection to complications such as hypoxemia, ARDS, and death. The combination of natriuretic and diuretic effects of these treatments may also limit pulmonary edema and protect the kidney from injury, a complication that develops in patients with severe disease. In addition, there is evidence that supports this approach to reduce the risk of coagulopathies. There is also evidence that these treatments may also protect the patient from the development of heart complications, such as myocarditis and acute cardiovascular syndrome.

Thus, new early drug interventions that protect patient populations at high risk of developing complications after COVID-19 infection, by restoring signaling through the GC-A receptor pathway to normal levels and preventing progression to pulmonary dysfunction, including, but not limited to, pulmonary edema and pulmonary inflammation, and subsequent associated complications, including hypoxemia, ARDS and death is another object of this invention. In some embodiments, pulmonary dysfunction is associated with or manifested as pulmonary edema. In other embodiments, pulmonary dysfunction is associated with or manifested as pulmonary inflammation.

In some embodiments, the interventions here contemplated may also protect the kidney from injury. In other embodiments, they may also protect the patient from the development of coagulopathies. In still other embodiments, they may also protect the patient from the development of heart complications, such as myocarditis and acute cardiovascular syndrome.

In some embodiments, the drug interventions here contemplated involve the use of an agent that increases signaling through the GC-A receptor. In other embodiments, the drug interventions here contemplated involve the use of an agent that increases intracellular cGMP through the GC-A pathway.

In some embodiments the agent is a NEP inhibitor. In other embodiments, the drug is a GC-A receptor agonist. In other embodiments, the intervention involves the use of both types of agents concomitantly. In some embodiments, the method further comprises administering to the patient one or more additional therapeutic agents other than the NEP inhibitor and/or the GC-A receptor agonist, or a pharmaceutically acceptable salt thereof. In certain embodiments, the additional therapeutic agents are those described herein. In some embodiments, the additional therapeutic agent is selected from a PDE5 inhibitor, a PDE9 inhibitor, NO, a NO donor, and an antiviral described herein.

NEP inhibitors that can be utilized in the methods, uses and compositions of this invention include, but are not limited to, sacubitril. In other embodiments, the NEP inhibitor is racecadotril, a prodrug or its active methabolite thiorphan. In other embodiments, the NEP inhibitor is ecadotril. In other embodiments the NEP inhibitor is TD-1439, TD-0714 or TD-0212 (Theravance compounds currently in clinical trials). In still other embodiments, the NEP inhibitor is selected from daglutril, ilepatril, SLV-338, UK-447841, VPN-489, LBQ657 or LHW-090.

Sacubitril is an inhibitor of the NEP enzyme that proteolytically degrades the natriuretic peptides atrial and brain natriuretic peptides (ANP and BNP, respectively) and thus elevates the plasma levels of these peptides. Elevated levels of natriuretic peptides lead to elevated levels of cGMP through activation of the GC-A receptor. Sacubitril is a component of the combination drug sacubitril-valsartan, known during clinical trials as LCZ696 and marketed in the US as Entresto. It is approved in the United States, Europe, and several other regions for the treatment of heart failure.

A NEP inhibitor alone, rather than in combination with an angiotensin receptor blocker (ARB, as is the case in the approved product Entresto) is the preferred drug. Valsartan (an ARB currently approved in combination with sacubitril for heart failure) would further lower blood pressure, which is already a complication in ARDS/Sepsis patients. Furthermore, in animal models, both angiotensin converting enzyme (ACE) inhibitors and angiotensin receptor blockers (ARBs) have been shown to upregulate angiotensin converting enzyme 2 (ACE2) receptor expression in the heart. Since ACE2 receptors have been shown to be the entry point into human cells for SARS-CoV-2, the virus that causes COVID-19, there are theoretical concerns that patients treated with ACE inhibitors or ARBs might be at increased risk for COVID-19 infection.

Treatment with Entresto has been shown to lead to increases in endogenous plasma levels of natriuretic peptides (ANP and BNP) and (plasma and urinary) cGMP in patients (Entresto prescribing information; McMurray, J. J. V., et al. “Angiotensin—Neprilysin Inhibition versus Enalapril in Heart Failure” N Engl J Med, 371, 993-1004, 2014).

Currently there are a number of other pharmacologic agents in clinical use that increase signaling through the GC-A-cGMP pathway. In one embodiment, the agents that can be utilized in the methods, uses and compositions of the invention are GC-A agonists. In some embodiments the GC-A agonist is ANP. In other embodiments, it is BNP. In still another embodiment, the agent is an ANP or BNP analogue.

There exist different forms of BNP that may be utilized in the methods, uses and compositions of the invention including, but not limited to, nesiritide.

Nesiritide is a synthetic form (recombinant form) of the human GC-A agonist BNP that was approved in the United States as a parenteral treatment for acute heart failure.

There exist different forms of ANP that may be utilized in the methods, uses and compositions of the invention including, but not limited to, carperitide.

Carperitide is a synthetic (recombinant) form of the GC-A agonist ANP that is approved in Japan for the treatment of acute heart failure (including acute exacerbation of chronic heart failure).

ANP or BNP analogues the can be utilized in the methods, uses and compositions of the invention, include but are not limited to orilotimod, PL-3994, TAK-639, ASB-20123, Fusion Protein to Agonize NPR2 from PhaseBio Pharmaceuticals (e.g., those described in WO2017/192449, US20130178416, and US20130143802), KTH-222, NPA-7, Peptides to Agonize NPR1 and NPR2 being developed by Oxydend Therapeutics, PHIN-1138, PL-5028 and several small molecule and synthetic peptides that agonize NPR2 from Daiichi Sankyo (e.g., those described in WO2013/161895, WO2015/129812, US2015/0125457, US2016/0017015, US2014/0072557, and WO2018/003983). ANP analgues that can be utilized in the methods, uses and compositions of the invention also include LA-ANP, which has amino acid sequence SLRRSSCFGGRMDRIGAQSGLGCNSFRYRITAREDKQGWA (SEQ ID NO:1) (described in patent application publication WO2007/035600 as SEQ ID No:2). ANP analogues that can be utilized in the methods, uses and compositions of the invention also include those described in patent application publication US2014/0066367, such as compound M-ANP)

Therapeutic Methods

In one aspect, the invention provides a method of treating the early stages of COVID-19 infection, comprising administering a therapeutically effective amount of a drug that increases signaling through the GC-A receptor, or a pharmaceutically acceptable salt thereof, to a patient in need thereof.

In another aspect, the invention provides a method of preventing the progression from the early stages of COVID-19 infection to hypoxemia, ARDS or death, comprising administering a therapeutically effective amount of a drug that increases signaling through the GC-A receptor, or a pharmaceutically acceptable salt thereof, to a patient in need thereof.

In another aspect, the invention provides a method of preventing the development of pulmonary dysfunction after developing early symptoms of COVID-19 infection, comprising administering a therapeutically effective amount of a drug that increases signaling through the GC-A receptor, or a pharmaceutically acceptable salt thereof, to a patient in need thereof. In some embodiments, pulmonary dysfunction is associated with or manifested as pulmonary edema. In other embodiments, pulmonary dysfunction is associated with or manifested as pulmonary inflammation.

In another aspect, the invention provides a method of preventing the progression to severe hypoxemia from mild hypoxemia in a patient with COVID-19, comprising administering a therapeutically effective amount of a drug that increases signaling through the guanylate cyclase-A (GC-A) receptor, or a pharmaceutically acceptable salt thereof, to a patient in need thereof.

In one aspect, the invention provides a method of treating the early stages of COVID-19 infection, comprising administering a therapeutically effective amount of a NEP inhibitor, or a pharmaceutically acceptable salt thereof to a patient in need thereof.

In another aspect, the invention provides a method of preventing the progression from the early stages of COVID-19 infection to hypoxemia, ARDS or death, comprising administering a therapeutically effective amount of a NEP inhibitor, or a pharmaceutically acceptable salt thereof to a patient in need thereof.

In another aspect, the invention provides a method of preventing the development of pulmonary dysfunction after developing early symptoms of COVID-19 infection, comprising administering a therapeutically effective amount of a NEP inhibitor, or a pharmaceutically acceptable salt thereof to a patient in need thereof. In some embodiments, pulmonary dysfunction is associated with or manifested as pulmonary edema. In other embodiments, pulmonary dysfunction is associated with or manifested as pulmonary inflammation.

In another aspect, the invention provides a method of preventing the progression to severe hypoxemia from mild hypoxemia in a patient with COVID-19, comprising administering a therapeutically effective amount of a NEP inhibitor, or a pharmaceutically acceptable salt thereof to a patient in need thereof.

In another aspect, the invention provides a method of treating the early stages of COVID-19 infection, comprising administering a therapeutically effective amount of a GC-A agonist or a pharmaceutically acceptable salt thereof to a patient in need thereof.

In another aspect, the invention provides a method of preventing the progression from the early stages of COVID-19 infection to hypoxemia, ARDS or death, comprising administering a therapeutically effective amount of a GC-A agonist or a pharmaceutically acceptable salt thereof to a patient in need thereof.

In another aspect, the invention provides a method of preventing the development of pulmonary dysfunction after developing early symptoms of COVID-19 infection, comprising administering a therapeutically effective amount of a GC-A agonist or a pharmaceutically acceptable salt thereof to a patient in need thereof. In some embodiments, pulmonary dysfunction is associated with or manifested as pulmonary edema. In other embodiments, pulmonary dysfunction is associated with or manifested as pulmonary inflammation.

In another aspect, the invention provides a method of preventing the progression to severe hypoxemia from mild hypoxemia in a patient with COVID-19, comprising administering a therapeutically effective amount of a GC-A receptor agonist or a pharmaceutically acceptable salt thereof to a patient in need thereof.

In another aspect, the invention provides pharmaceutical compositions comprising a NEP inhibitor, a GC-A agonist or a pharmaceutically acceptable salt thereof, for use in method for treating the early stages of COVID-19 infection, preventing the progression from the early stages of COVID-19 infection to hypoxemia, ARDS or death, preventing the development of pulmonary dysfunction after developing early symptoms of COVID-19 infection or preventing the progression from mild hypoxemia to severe hypoxemia in a patient in need thereof. Also included is the use of a NEP inhibitor, a GC-A agonist or a pharmaceutically acceptable salt thereof, for the manufacture of a medicament for treating the early stages of COVID-19 infection, preventing the progression from the early stages of COVID-19 infection to hypoxemia, ARDS or death, preventing the development of pulmonary dysfunction or preventing the progression from mild hypoxemia to severe hypoxemia after developing early symptoms of COVID-19 infection in a patient in need thereof. In some embodiments of the above pharmaceutical compositions, pulmonary dysfunction is associated with or manifested as pulmonary edema. In other embodiments, pulmonary dysfunction is associated with or manifested as pulmonary inflammation.

In some embodiments, of the above methods, uses and compositions, the treatment also results in protection of the kidney from injury.

In other embodiments, they may also protect the patient from the development of coagulopathies.

In still other embodiments, these treatments may also protect the patient from the development of heart complications, such as myocarditis and acute cardiovascular syndrome.In some embodiments, for methods, uses and pharmaceutical compositions for use described herein, the NEP inhibitor and the GC-A agonist may be used concomitantly.

The term “disorder”, as used herein refers to any deviation from or interruption of the normal structure or function (dysfunction) of any body part, organ, or system that is manifested by a characteristic set of symptoms and signs and whose etiology, pathology, and prognosis may be known or unknown. The term disorder encompasses other related terms such as “disease” and “condition” (or medical condition) as well as syndromes, which are defined as a combination of symptoms resulting from a single cause or so commonly occurring together as to constitute a distinct clinical picture. In some embodiments, the term disorder refers to COVID-19 infection or any of its complications. In some embodiments, complications of COVID-19 infection comprise the development of lung dysfunction, hypoxemia, respiratory distress, ARDS or death.

“Pulmonary dysfunction” can be manifested in many different ways, which are known by persons of skill in the art, such as doctors, clinicians, researchers, etc. Manifestations include but are not limited to inflammation or edema of the lungs. In some embodiments, pulmonary dysfunction may manifest as pulmonary edema. In other instances, it may manifest as pulmonary inflammation.

“Acute Respiratory Distress Syndrome” (ARDS) is a medical condition in which fluid builds up in the alveoli of the lungs. The fluid keeps the lungs from filling with enough air, which results in less oxygen reaching the bloodstream. This deprives the organs of the oxygen they need to function.

The mechanical cause of ARDS is fluid leaked from the smallest blood vessels in the lungs into the alveoli, where blood is oxygenated. Normally, a protective membrane of endothelial tissue keeps this fluid in the vessels. However, severe illness or injury can cause damage to the membrane, increasing its permeability and leading to the fluid leakage of ARDS. The SARS-CoV2 coronavirus damages both the wall and lining cells of the alveolus as well as the capillaries including the endothelium. The damage to capillaries also causes them to leak plasma proteins that add to the wall's thickness. Eventually, the wall of the alveolus gets thicker than it should be. The thicker this wall gets, the harder it is to transfer oxygen

The signs and symptoms of ARDS can vary in intensity, depending on its cause and severity, as well as the presence of underlying heart or lung disease. They include, but are not limited to severe shortness of breath, labored and unusually rapid breathing, low blood pressure, confusion and extreme tiredness.

ARDS typically occurs in people who are already critically ill or who have significant injuries. Severe shortness of breath—the main symptom of ARDS—usually develops within a few hours to a few days after the precipitating injury or infection.

To date, there have been limited numbers of descriptions of the lung pathophysiology associated with ARDS in COVID-19 disease. In a case report of a 50-year old patient with COVID-19 who developed ARDS and died (Xu, Z., et al. “Pathological findings of COVID-19 associated with acute respiratory distress syndrome” Lancet Respiratory Medicine, published online, Feb. 18, 2020), the right lung showed evident desquamation of pneumocytes and hyaline membrane formation, indicating ARDS. The left lung tissue displayed pulmonary edema with hyaline membrane formation, suggestive of early-phase ARDS. Interstitial mononuclear inflammatory infiltrates, dominated by lymphocytes, were seen in both lungs. Multinucleated syncytial cells with atypical enlarged pneumocytes characterized by large nuclei, amphophilic granular cytoplasm, and prominent nucleoli were identified in the intra-alveolar spaces, showing viral cytopathic-like changes. No obvious intranuclear or intracytoplasmic viral inclusions were identified. These pathological features of COVID-19 greatly resemble those seen in a 2003 outbreak of SARS (Ding et al “The clinical pathology of severe acute respiratory syndrome (SARS): a report from China” J Pathol. 200(3): 282-9, 2003) where pulmonary lesions included bilateral extensive consolidation, localized haemorrhage and necrosis, desquamative pulmonary alveolitis and bronchitis, proliferation and desquamation of alveolar epithelial cells, exudation of protein and monocytes, lymphocytes and plasma cells in alveoli, hyaline membrane formation, and viral inclusion bodies in alveolar epithelial cells. The lesions' features also resembled those in MERS outbreak of 2014.

In some embodiments, for methods, uses and compositions of the present invention, the precipitating infection is COVID-19. In some embodiments of the above methods, uses and compositions, the precipitating injury to the lungs is the result of COVID-19 infection.

The risk of death after developing ARDS increases with age and severity of illness as well as the presence of concomitant or pre-existing conditions. Underlying CV disease is one of such risk factors. More specifically, the presence of hypertension has been observed to increase the risk of developing complications form COVID-19 infection. Of the people who do survive ARDS, some recover completely while others experience lasting damage to their lungs.

In a report based on a Wuhan cohort of COVID-19 patients it was found that 31% of patients developed ARDS and 93% of those patients died (Zhou, F., et al “Clinical course and risk factors for mortality of adult inpatients with COVID-19 in Wuhan, China: a retrospective cohort study.” Lancet 395(10229), 1054-1062, 2020).

In some embodiments, for methods, uses and compositions of the present invention described herein, treatment with a NEP inhibitor or a GC-A agonist or a combination thereof results in a reduced incidence of death. In other embodiments, it results in a reduction in the number or length of hospitalizations (for example, number of hospitalizations, or total days of hospitalization, per number of patients).

“Hypoxemia”, as used herein, is a state in which oxygen is present in below-normal levels in blood, specifically in the arteries. It is a sign of a problem related to breathing or circulation, and may result in various symptoms, such as shortness of breath or laboring breathing (dyspnea). Levels of oxygen in blood can be measured by pulse oximetry.

When patients develop ARDS, and the accompanying hypoxemia, they may necessitate the use of a ventilator to assist with breathing. Estimates so far show that about 6% of people who have COVID-19 get critically sick. And about 1 in 4 of them may need a ventilator to help them breathe, with the picture changing quickly as the infection continues to spread around the globe.

In the current COVID-19 pandemic, the increased need for ventilator use and shortage of enough units thereof has become, in many countries, one of the reasons for overwhelming the medical systems and crisis. It is expected to lead to medical system collapse in some countries and regions.

In some embodiments, for methods, uses and compositions of the present invention described herein, treatment with a NEP inhibitor or a GC-A agonist results in decreased need for ventilator use in patients with COVID-19. In situations in which there are not enough ventilators, this would, in turn, lead to an improvement in the outcome of death.

Some underlying conditions in COVID-19 patients that may trigger ARDS include, but are not limited to, sepsis (serious and widespread infection of the bloodstream) and severe pneumonia, which usually can affect all five lobes of the lungs.

Some pre-existing conditions that make COVID-19 ARDS patients more likely to progress to ventilator use or death include, but are not limited to heart disease, hypertension, obesity and diabetes.

Some additional factors that make COVID-19 ARDS patients form likely to progress to ventilator use or death includes elevated age.

In some embodiments, for methods, uses and compositions of the present invention described herein, the NEP inhibitor or GC-A agonist is administered before symptoms of COVID-19 fully develop in said patient. In some embodiments, the patient has given a positive result after taking a COVID-19 test. In some embodiments the patient is one that is considered to be at risk of developing pulmonary dysfunction after developing early COVID-19 symptoms or giving a positive result in a COVID-19 test. In other embodiments, the patient is showing early symptoms of COVID-19. In some embodiments, early symptoms of COVID-19 comprise fever, body aches or tiredness. In some embodiments, early symptoms of COVID-19 comprise dry cough. In other embodiments, early symptoms of COVID-19 comprise dyspnea.

In other embodiments, for methods, uses and compositions of the present invention described herein, the NEP inhibitor or GC-A receptor agonist is administered after one or more symptoms of lung dysfunction develops in said patient. In some embodiments, symptoms of lung dysfunction comprise severe breathing difficulties, hypoxemia, confusion, tiredness.

In other embodiments, for methods, uses and compositions of the present invention described herein, the NEP inhibitor or GC-A receptor agonist is administered after the patient develops ARDS.

In some embodiments, the patient is treated in a hospital setting. In other embodiments, the patient has not been hospitalized and is treated at home or in another community setting, such as an elderly residence.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In some embodiments, the subject is a non-human animal such as a farm animal (e.g., a horse, cow, pig or sheep), or a companion animal or pet (e.g., a dog, cat, mice, rats, hamsters, gerbils, guinea pig or rabbit). In some embodiments, the subject is a human.

In some embodiments, the “patient in need thereof” is a patient with COVID-19 or who has been diagnosed with it or who is known to have been in contact with a confirmed COVID-19 patient and has developed some early symptoms of the disease. In some embodiments, the patient can be confirmed to have COVID-19 either by a clinical test (e.g. a blood test, a chest CT scan, etc) or by a set of clinical symptoms/signs or by a combination thereof. In some embodiments of the above methods, uses and compositions, the patient in need thereof is an adult. In other embodiments the patient is a child. In still other embodiments the patient in need thereof is an infant. In still other embodiments, the patient is an adult over 70 years old. In yet other embodiments the patient is an adult over 60 years old. In other embodiments the patient is an adult over 80 years old.

In one embodiment, the patient in need thereof is a patient belonging to a population at high risk of complications after COVID-19 infection. In some embodiments, the patient in need thereof is a patient that is 65 or older. In another embodiment, the patient is 70 years old or older. In another embodiment, the patient is 80 years old or older.

In another embodiment, the patient in need therof is a patient that is diagnosed to have hypertension. In some embodiments, the patient is receiving treatment for hypertension with one or more anti-hypertensives. In other embodiments, the hypertension is well controlled by treatment with one or more anti-hypertensives. In still other embodiments, the hypertension is not well controlled despite treatment with one or more anti-hypertensives. In yet other embodiments, the patient with hypertension is receiving treatment with an ARB inhibitor and/or an ACE inhibitor.

In another embodiment, the patient in need thereof is a patient in a population at high risk of complications after COVID-19 infection because of its race or ethnic origin. In one embodiment, the patient is not of Hispanic or Latino ethnicity. In another embodiment, the patient is of Hispanic or Latino ethnicity. In some of these two embodiments, the patient is non-white. In other embodiments, the patient is black or African American. In other embodiments the patient is an American Indian or an Alaska native. In other embodiments, the patient is an Asian. In still other embodiments the patient is a Native Hawaiian or a Pacific Islander.

In another embodiment, the patient in need thereof is considered a patient in a population at high risk of complications after COVID-19 infection because he/she is a diabetic. In some embodiments, the patient is a pre-diabetic. In still other embodiments, the patient's glucose levels are well controlled by anti-hyperglycemic treatment. In other embodiments, the patient's glucose levels are not well controlled despite treatment with one or more anti-hyperglycemic drugs.In another embodiment, the patient in need thereof is considered a patient in a population at high risk of complications after COVID-19 infection because he/she is overweight. In other embodiments, the patient is obese. In still other embodiments, the patient is morbid obese.

In some embodiments, the patient shows symptoms of pulmonary dysfunction. In some of these embodiments, pulmonary dysfunction is associated with or manifested as pulmonary edema. In other embodiments, pulmonary dysfunction is associated with or manifested as pulmonary inflammation.

In some embodiments, the patient has developed kidney injury or damage after being infected by SARS-CoV-2 and before the initiation of treatment with the interventions of the invention.

In other embodiments, the patient has developed a coagulopathy after being infected by SARS-CoV-2 and before the initiation of treatment with the interventions of the invention.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disorder or one or more of its symptoms, or to prevent or substantially lessen the chances of acquiring a disorder or a symptom or to reduce the severity of the disorder or one or more of its symptoms before it is acquired or before the symptoms develop further or fully develop. Therapeutically effective amounts of approved drugs are disclosed in their labels or can be determined by persons of the skill in the art.

In certain embodiments, when nesiritide is utilized in the methods, uses and compositions of the present invention, it can be administered to the patient by an intravenous bolus injection of 2 mcg/kg followed by a continuous infusion of 0.01 mcg/kg/min.

In certain embodiments, when Entresto is used in the methods of the present invention, a dosage between 49 mg and 97 mg can be used for an adult patient.

The compounds and pharmaceutical compositions described herein can be used alone or in combination therapy with additional therapeutic agents.

“Treat”, “treating” or “treatment” with regard to a disorder, disease, condition, symptom or syndrome, refers to abrogating or improving the cause and/or the effects (i.e., the symptoms, physiological or physical manifestations) of the disorder, disease, condition or syndrome. As used herein, the terms “treat”, “treatment” and “treating” also refer to the delay or amelioration or prevention of the progression (i.e. the known or expected progression of the disease), severity and/or duration of the disease or delay or amelioration or prevention of the progression of one or more symptoms (i.e. “managing” without “curing” the condition), resulting from the administration of one or more therapies.

Pharmaceutical Compositions and Methods of Administration

The compounds herein disclosed, and their pharmaceutically acceptable salts thereof may be formulated as pharmaceutical compositions or “formulations”.

A typical formulation is prepared by mixing a therapeutically active agent, or a pharmaceutically acceptable salt thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the active agent is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., NEP inhibitor, ANP, BNP or a pharmaceutical composition thereof) or aid in the manufacturing of the pharmaceutical product (i.e., medicament).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

The formulations may be prepared using conventional dissolution and mixing procedures.

The terms “administer”, “administering” or “administration” in reference to a compound, composition or dosage form of the invention means introducing the compound into the system of the subject or patient in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g. orally (including, but not limited to solid dosage forms including hard or soft capsules (e.g. gelatin capsules), tablets, pills, powders, sublingual tablets, troches, lozenges, and granules; and liquid dosage forms including, but are not limited to, pharmaceutically acceptable emulsions, microemulsions, aqueous or oil solutions, suspensions, syrups and elixirs, by inhalation (e.g. with an aerosol, gas, inhaler, nebulizer or the like), to the ear (e.g. using ear drops), topically (e.g. using creams, gels, inhalants, liniments, lotions, ointments, patches, pastes, powders, solutions, sprays, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via ear drops, via an implanted reservoir or the like, or parenterally depending on the severity and type of the disorder being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Formulations of a compound intended for oral use may be prepared according to any method known to the art for the manufacture of pharmaceutical compositions.

In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The oral compositions (either solid or liquid) can also include excipients and adjuvants such as dispersing or wetting agents, such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate); emulsifying and suspending agents, such as sodium carboxymethylcellulose, croscarmellose, povidone, methylcellulose, hydroxypropyl methylcelluose, sodium alginate, polyvinylpyrrolidone, gum tragacanth and gum acacia; sweetening, flavoring, and perfuming agents; and/or one or more preservatives such as ethyl or n-propyl p-hydroxy-benzoate, one or more coloring agents, one or more flavoring agents and one or more sweetening agents, such as sucrose or saccharin.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc.) which is administered by rapid inhalation through the nasal passage or by inhalation through the mouth so as to reach the alveolar sacs.

The pharmaceutical compositions described herein may also be administered topically, especially when the target of treatment includes areas or organs readily accessible by topical application, including disorders of the eye, the ear, the skin, or the lower intestinal tract. Suitable topical formulations are readily prepared for each of these areas or organs. The active component is admixed under sterile conditions with a pharmaceutically acceptable carrier and any needed preservatives or buffers as may be required.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using a therapeutic agent of the inventionmay be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of a an therapeutic agent of the invention include Tween™-60, Span™-80, cetostearyl alcohol, benzyl alcohol, myristyl alcohol, glyceryl mono-stearate and sodium lauryl sulfate.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

Sterile injectable forms of the compositions described herein (e.g. for parenteral administration) may be aqueous or oleaginous suspension. These suspensions may be formulated according to techniques known in the art using suitable dispersing or wetting agents and suspending agents (including those described in the preceding paragraph). The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as a vegetable oil, for example arachis oil, olive oil, sesame oil or coconut oil, especially in their polyoxyethylated versions, or in mineral oil such as liquid paraffin. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

In another aspect, a an therapeutic agent of the invention or a pharmaceutically acceptable salt thereof may be formulated in a veterinary composition comprising a veterinary carrier. Veterinary carriers are materials useful for the purpose of administering the composition and may be solid, liquid or gaseous materials which are otherwise inert. In the veterinary art and are compatible with the active ingredient. These veterinary compositions may be administered parenterally, orally or by any other desired route.

Combination Therapies

As used herein, the terms “in combination” or “co-administration” can be used interchangeably to refer to the use of more than one therapy. The use of the terms does not restrict the order in which therapies are administered to a subject.

In some embodiments, the therapeutic agent of the invention is administered prior to, at the same time or after the initiation of treatment with another therapeutic agent.

In some embodiments, for methods, uses and compositions of the present invention described herein, the additional therapeutic agent and the therapeutic agent of the invention are administered simultaneously. In other embodiments, for methods, uses and compositions of the present invention described herein, the additional therapeutic agent and the therapeutic agent of the invention are administered sequentially or separately.

In addition, the administration of one agent may be prior to, concurrent to, or subsequent to the administration of the other agent.

When used in combination therapy with other agents, a “therapeutically effective amount” of the therapeutic agent of the invention and of the other agent or agents will depend on the type of drug used. Suitable dosages are known for approved agents and can be adjusted by the skilled artisan according to the condition of the subject or the type of condition(s) being treated. In cases where no amount is expressly noted, an effective amount should be assumed.

When co-administration involves the separate administration of a first amount of a therapeutic agent of the invention and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, the therapeutic agent of the invention and the second (or subsequent) therapeutic agent(s) can be administered in any order within about 24 hours of each other, within about 16 hours of each other, within about 8 hours of each other, within about 4 hours of each other, within about 1 hour of each other, within about 30 minutes of each other, within 15 minutes of each other.

Examples of other therapeutic agents that may be combined with therapeutic agent of the invention, or a pharmaceutically acceptable salt thereof, include, but are not limited to:

(1) Endothelium-derived releasing factor (EDRF) or NO gas.

(2) NO donors such as a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodium nitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; an m-nitroxymethyl phenyl ester of acetyl salicylic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; isosorbide (Dilatrate®-SR, Imdur®, Ismo®, Isordil®, Isordil®, Titradose®, Monoket®), 3-morpholinosydnonimine; linsidomine chlorohydrate (“SIN-1”); S-nitroso-N-acetylpenicillamine (“SNAP”); S-nitrosoglutathione (GSNO), sodium nitroprusside, S-nitrosoglutathione mono-ethyl-ester (GSNO-ester), 6-(2-hydroxy-1-methyl-nitrosohydrazino)-N-methyl-1-hexanamine or diethylamine NONOate.

(3) Other substances that enhance cGMP concentrations such as protoporphyrin IX, arachidonic acid and phenyl hydrazine derivatives.

(4) Nitric Oxide Synthase substrates: for example, L-arginine, n-hydroxyguanidine based analogs, such as N[G]-hydroxy-L-arginine (NOHA), 1-(3,4-dimethoxy-2-chlorobenzylideneamino)-3-hydroxyguanidine, and PR5 (1-(3,4-dimethoxy chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluoromethyl) propylguanidine.

(5) Compounds which enhance eNOS transcription.

(6) NO independent heme-independent sGC activators, including, but not limited to:

BAY 58-2667 (described in patent publication DE19943635); HMR-1766 (ataciguat sodium, described in patent publication WO2000002851);S 3448 (2-(4-chloro-phenylsulfonylamino)-4,5-dimethoxy-N-(4-(thiomorpholine-4-sulfonyl)-phenyl)- benzamide (described in patent publications DE19830430 and WO2000002851); and

HMR-1069 (Sanofi-Aventis).

(7) Heme-dependent, NO-independent sGC stimulators including, but not limited to:

YC-1 (see patent publications EP667345 and DE19744026); riociguat (BAY 63-2521, Adempas®, described in DE19834044); neliciguat (BAY 60-4552, described in WO 2003095451); vericiguat (BAY 1021189); BAY 41-2272 (described in DE19834047 and DE19942809); BAY 41-8543 (described in DE19834044); etriciguat (described in WO 2003086407); CFM-1571 (described in patent publication WO2000027394); A-344905, its acrylamide analogue A-350619 and the aminopyrimidine analogue A-778935;

other sGC stimulators described in one of publications US20090209556, US8455638, US20110118282 (WO2009032249), US20100292192, US20110201621, US7947664, US8053455 (WO2009094242), US20100216764, US8507512, (WO2010099054) US20110218202 (WO2010065275), US20130012511 (WO2011119518), US20130072492 (WO2011149921), US20130210798 (WO2012058132) and Tetrahedron Letters (2003), 44(48): 8661-8663; and

IW-1973 and IW1701.

(8) Compounds that inhibit the degradation of cGMP and/or cAMP, such as:

PDE1 inhibitors, PDE2 inhibitors, PDE-3 inhibitors such as, for example, amrinone, milrinone, enoximone, vesnarinone, pimobendan, and olprinone, PDE4 inhibitors, such as, for example, rolumilast, PDE5 inhibitors, such as, for example, sildenafil (Viagra®) and related agents such as avanafil, lodenafil, mirodenafil, sildenafil citrate (Revatio®), tadalafil (Cialis° or Adcirca®), vardenafil (Levitra®) and udenafil; alprostadil; dipyridamole and PF-00489791; PDE6 inhibitors, PDE9 inhibitors, such as, for example, PF-04447943; osoresnontrine, E-2027, tovinontrine, CRD-733 or TT920, PDE10 inhibitors such as, for example, PF-02545920 (PF-10), and PDE11 inhibitors.

(9) antivirals such as the combination of emtricitabine with tenofovir disoproxil fumarate, oseltamivir or salts thereof, laninamivir or salts thereof, baloxavir, darunavir, cobicistat, lopinavir, ritonavir, remdesivir, favipiravir, chloroquine and peramivir.

(10) Anticoagulants, including but not limited to:

coumarines (Vitamin K antagonists) such as warfarin, cenocoumarol, phenprocoumon and phenindione;

heparin and derivatives such as low molecular weight heparin, fondaparinux and idraparinux;

direct thrombin inhibitors such as argatroban, lepirudin, bivalirudin, dabigatran and ximelagatran; and

tissue-plasminogen activators, used to dissolve clots and unblock arteries, such as alteplase.

Packaging and Kits

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the disorders described herein). The kit may optionally comprise a second pharmaceutical composition comprising one or more additional agents described herein for co therapy use, a pharmaceutically acceptable carrier, vehicle or diluent. The pharmaceutical composition comprising the compound described herein and the second pharmaceutical composition contained in the kit may be optionally combined in the same pharmaceutical composition.

### EXAMPLES

ANP and other agents that increase cGMPin endothelial cells have been found to block the increase of permeability in the endothelium, wherein the permeability arises from a number of causes (Baron D. A., et al, “Atriopeptin Inhibitionof Thrombin-Mediated Changes in the Morphology and Permeability of Endothelial Monolayers” Proc Natl Acad Sci USA, 86(9), 3394-8, 1989; Lofton C.E., et al. “Atrial Natriuretic Peptide Regulation of Endothelial Permeability Is Mediated by cGMP” Biochem Biophys Res Commun, 172(2), 793-9,1990). Critically, this mechanism is cyclic GMP dependent and appears to universally prevent all drivers of increased vascular permeability, particularly in the lung (Lofton C. E., et al. 1990).

Exogenous administration a synthetic form of the GC-A agonist peptide atrial natriuretic peptide ANP has been shown to protect from lung injury and endothelial barrier dysfunction in all a number of experimental noxious conditions. For instance, ANP attenuated pulmonary oedema induced by congestive heart failure in dogs (Riegger G. A., et al., “Effects of ANP-(95-126) in dogs before and after induction of heart failure” Am J Physiol , 259(6 pt 2): H1643-8, 1990) or by lung ischaemia-reperfusion injury in rodents (Dodd-o J M, et al. “The role of natriuretic peptide receptor-A signaling in unilateral lung ischemia-reperfusion injury in the intact mouse.” Am JPhysiol Lung Cell Mol Physiol 294: L714—L723, 2008). In mice, ANP pretreatment protected against lung injury, inflammation and endothelial barrier dysfunction induced by gram-negative bacterial wall LPS or Staphylococcus Aureus infection (Birukova A A, et al. “Atrial natriuretic peptide attenuates LPS-induced lung vascular leak: role of PAK1.” Am JPhysiol 299: L652—L663, 2010; Xing J, Birukova A A “ANP attenuates inflammatory signaling and Rho pathway of lung endothelial permeability induced by LPS and TNFalpha.” Microvasc Res 79: 56-62, 2010).

Clinical studies support the therapeutic relevance of these experimental observations: intravenous ANP infusion improved pulmonary gas exchange and the lung injury score in patients with acute lung injury during mechanical ventilation with positive end-expiratory pressure (Mitaka C, Hirata Y, Nagura T, Tsunoda Y, Amaha K “Beneficial effects of atrial natriuretic peptide on pulmonary gas exchange in patients with acute lung injury.” Chest 114: 223-228, 1998) and diminished pulmonary oedema and pulmonary vascular permeability in intensive care patients without heart disease (Sakamoto Y, et al. “Effectiveness of human atrial natriuretic peptide supplementation in pulmonary edema patients using the pulse contour cardiac output system.” Yonsei Med J 51: 354-359, 2010). Lentiviral overexpression of GC-A enhanced the protective effect of ANP in a model of acute lung injury induced by aspiration of Pseudomonas aeruginosa (Friebe A., et al. “cGMP: a unique 2nd messenger molecule—recent developments in cGMP research and development” Naunyn-Schmiedeberg's Archives of Pharmacology 393, 287-302, 2020).

In an experimental and clinical study of acute lung injury (ALI)/ARDS, the activity of NEP was significantly decreased in plasma and increased in the alveolar air space (Hashimoto S, Amaya F, Oh-Hashi K, Kiuchi K, Hashimoto S “Expression of neutral endopeptidase activity during clinical and experimental acute lung injury” Respir Res. 11:164, 2010).

Mice with targeted disruption of the gene encoding the ANP-degrading enzyme neutral endopeptidase (NEP24.11) showed a greater relative rise in plasma ANP levels, attenuated pulmonary vascular pressure and reduced pulmonary vascular albumin and fluid leak during high altitude exposure (Irwin D C, Patot M T, Tucker A, Bowen R (2005b). “Neutral endopeptidase null mice are less susceptible to high altitudeinduced pulmonary vascular leak.” High Alt Med Biol 6: 311-319, 2005).

In edema provoked by infections of the lung or hypoxia, the protective ANP effects could be mediated by the GC-A receptor on inflammatory cells such as macrophages, mast cells or neutrophils (Opgenorth T J, Budzik G P, Mollison K W, Davidsen S K, Holst M R, Holleman W H (1990). Atrial peptides induce mast cell histamine release. Peptides 11: 1003-1007; Wiedermann C J, Niedermalbichler M, Braunsteiner H, Widermann C J (1992). Priming of polymorphonuclear neutrophils by atrial natriuretic peptide in vitro. J Clin Invest 89: 1580-1586; Vollmar A M, Förster R, Schulz R (1997). Effects of atrial natriuretic peptide on phagocytosis and respiratory burst in murine macrophages. Eur J Pharmacol 319: 279-285.; Kiemer A K, Vollmar A M (2001). The atrial natriuretic peptide regulates the production of inflammatory mediators in macrophages. Ann Rheum Dis 60 (Suppl. 3): iii68—iii70; Tsukagoshi H, Shimizu Y, Kawata T, Hisada T, Shimizu Y, Iwamae S et al. (2001). Atrial natriuretic peptide inhibits tumor necrosis factor-alpha production by interferon-gamma-activated macrophages via suppression of p38 mitogen-activated protein kinase and nuclear factor-kappa B activation. Regul Pept 99: 21-29).

Indicating a direct action on the pulmonary vascular bed, various studies have shown that ANP has a direct anti-oedematic action in isolated perfused lung models subjected to increased capillary pressure, hypoxia or inflammatory stimuli such as detergents or prostaglandins (Inomata N, Ohnuma N, Furuya M, Hayashi Y, Kanai Y, Ishihara T et al. (1987). Alpha-human atrial natriuretic peptide (alpha-HANP) prevents pulmonary edema induced by arachidonic acid treatment in isolated perfused lung from guinea pig. Jpn J Pharmacol 44: 211-214; Imamura T, Ohnuma N, Iwasa F, Furuya M, Hayashi Y, Inomata N et al. (1988). Protective effect of alpha-human atrial natriuretic polypeptide (alpha-HANP) on chemical-induced pulmonary edema. Life Sci 42: 403-414). Yet, these studies could not distinguish whether the attenuation of interstitial oedema was due to the ANP-provoked vasodilatation, decreasing pulmonary capillary hydrostatic pressure, or to direct endothelial barrier-enhancing (stabilizing) effects.

Suggesting the participation of a direct endothelial protective effect, ANP reduced hypoxia, TNF-a, thrombin, or bacterial endotoxin (LPS)—induced paracellular hyperpermeability of pulmonary microvascular and macrovascular endothelial cells cultured on permeable supports (Lofton C E, Baron D A, Heffner J E, Currie M G, Newman W H (1991). “Atrial natriuretic peptide inhibits oxidant-induced increases in endothelial permeability.” J Mol Cell Cardiol 23: 919-927; Irwin D C, Tissot van Patot M C, Tucker A, Bowen R (2005). “Direct ANP inhibition of hypoxia-induced inflammatory pathways in pulmonary microvascular and macrovascular endothelial monolayers.” Am J Physiol Lung Cell Mol Physiol 288: L849—L859; Klinger J R, Warburton R, Carino G P, Murray J, Murphy C, Napier M et al. (2006). “Natriuretic peptides differentially attenuate thrombin-induced barrier dysfunction in pulmonary microvascular endothelial cells.” Exp Cell Res 312: 401-410; Scott R S, Rentsendorj O, Servinsky L E, Moldobaeva A, Damico R, Pearse D B (2010) “Cyclic GMP increases antioxidant function and attenuates oxidant cell death in mouse lung microvascular endothelial cells by a protein kinase G-dependent mechanism.” Am J Physiol Lung Cell Mol Physiol 299: L323-L333).

In clinical studies, the combination of a PDE5 inhibitor (PDE5i) with a neprilysin inhibitor augmented natriuretic peptide bioactivity, promoted cGMP signalling, and reversed the structural and haemodynamic deficits that characterize pulmonary arterial hypertension (PAH). For example, in a clinical trial in patients with pulmonary arterial hypertension, treatment with the the neprilysin inhibitor racecadotril resulted in a 79% increase in the plasma ANP concentration and a 106% increase in plasma cGMP levels with a concomitant 14% reduction in pulmonary vascular resistance (Hobbs, A. J. et al. “Neprilysin inhibition for pulmonary arterial hypertension: a randomized, double-blind, placebo-controlled, proof-of-concept trial”, Br J Pharmacol 176(9):1251-1267 2019).

Lending additional support to the therapeutic hypothesis of cGMP modulation in COVID-19, an approach using inhaled nitric oxide (NO) is currently under investigation in 6 clinical trials in COVID-19 patients (clinical trials.gov: NCT04383002, NCT04338828, NCT04358588, NCT04305457, NCT04305457, NCT04337918, NCT04306393).

Taken together, these numerous studies in vitro/in vivo indicate that ANP, at least when given as exogenous pharmacological agent, exerts endothelial barrier-protecting actions in the pulmonary circulation and could be effective in the protection of pulmonary function in COVID-19 patients. They also indicated that NEP inhibition may be an effective method to increase ANP concentrations in vivo.

### Example 1

In order to test the efficacy of these treatments in COVID-19 patients, a placebo-controlled trial would evaluate the 2-4 week treatment impact of any of the above therapeutic agents of the invention on the need for ventilators (for example by assessing number of days on ventilator), hospitalization length (for example by assessing time to hospital discharge), ICU use (for example by assessing number of days in ICU), incidence of death (for example by assessing number of deaths per number of confirmed COVID-19 patients), effects on kidney protection (for example by assessing level of kidney function at hospitalization and comparing it with kidney function at time of release), heart protection (comparing the level of heart function at hospitalization with that at time of release). The trial would initially enroll non-hospitalized symptomatic patients with either dry cough or mild dyspnea. The trial may have several arms including, for example, patients on placebo, patients receiving a NEP inhibitor, patients receiving a GC-A agonist or patients receiving a combination of both agents. Further trials could enroll patients that are in earlier stages of the disease (e.g. early mild symptoms and confirmed COVID-19 by any accepted method and preexisting known risks such as age or preexisting conditions, such as obesity, diabetes, and hypertension, or early mild symptoms and known to have been in close contact with a confirmed case and known risks).

### Example 2

In another clinical trial, a natriuretic hormone (ANP or BNP), for example an IV infusion of nesiritide, would be used. In this case, the trial would enroll patients that have been admitted into the intensive care unit (ICU) as a result of the COVID-19 infection and are showing signs of worsening hypoxemia. The treatments proposed may prevent the progression of the disease from mild to severe hypoxemia, or further to ARDS, and death.

This trial may include several arms, such as placebo, natriuretic hormone treatment alone and maybe natriuretic hormone treatment in combination with other agents.

For both Examples, the highest marketed dose of each of the agents would be an immediate approach to test the potential of this therapeutic approach. But other dosages could be tested as necessary and persons of skill in the art would be able to determine appropriate dosage regimens in each case.

### OTHER EMBODIMENTS

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims. A number of embodiments have been described. Nevertheless, it will be understood that various modifications may be made without departing from the spirit and scope of the invention.

We claim:

1. A method of treating the early stages of COVID-19 infection in a patient in need thereof, comprising administering to the patient a therapeutically effective amount of a neural endopeptidase (NEP) inhibitor, or a pharmaceutically acceptable salt thereof.

2. The method of claim 1, wherein the method prevents the progression from the early stages of COVID-19 infection to hypoxemia, acute respiratory distress syndrome (ARDS) or death.

3. The method of claim 1, wherein the method prevents the development of pulmonary dysfunction after developing early symptoms of COVID-19 infection.

4. The method of claim 1, wherein the method prevents the progression from mild hypoxemia to severe hypoxemia in a patient with COVID-19.

5. The method of any one of claims 1-4, wherein the NEP inhibitor is selected from sacubitril, TD-1439, TD-0714, TD-0212, daglutril, ilepatril, SLV-338, UK-447841, VPN-489, LBQ657 and LHW-090.

6. A method of treating the early stages of COVID-19 infection in a patient in need thereof, comprising administering to the patient a therapeutically effective amount of a guanylate cyclase-A (GC-A) receptor agonist, or a pharmaceutically acceptable salt thereof.

7. The method of claim 6, wherein the method prevents the progression from the early stages of COVID-19 infection to hypoxemia, ARDS or death.

8. The method of claim 6, wherein the method prevents the development of pulmonary dysfunction after developing early symptoms of COVID-19 infection.

9. The method of claim 8, wherein pulmonary dysfunction is pulmonary edema.

10. The method of claim 9, wherein pulmonary dysfunction is pulmonary inflammation.

11. The method of claim 6, wherein the method prevents the progression from mild hypoxemia to severe hypxemia in a patient with COVID-19.

12. The method of claim 6, wherein the method protects the kidney from injury.

13. The method of claim 6, wherein the method protects the patient from the development of coagulopathies.

14. The method of claim 6, wherein the method protects the patient from the development of heart complications.

15. The method of claim 14, wherein the heart complications are selected from myocarditis and acute cardiovascular syndrome.

16. The method of any one of claims 6-15, wherein the GC-A receptor agonist is selected from an atrial natriuretic peptide (ANP), an ANP analogue, a brain natriuretic peptide (BNP) and a BNP analogue.

17. The method of claim 16, wherein the GC-A receptor agonist is selected from nesiritide, carperitide, orilotimod, PL-3994, TAK-639, ASB-20123, Fusion Protein to Agonize NPR2 from PhaseBio Pharmaceuticals, KTH-222, NPA-7, Peptide to Agonize NPR1 and NPR2 from Oxydend Therapeutics, PHIN-1138, PL-5028, several small molecule and synthetic peptides that agonize NPR2 from Daiichi Sankyo and M-ANP.

18. The method of any one of claims 6-17, wherein the method further comprises administering to the patient a therapeutically effective amount of a NEP inhibitor, or a pharmaceutically acceptable salt thereof.

19. The method of claim 18, wherein the NEP inhibitor and the GC-A receptor agonist are administered concomitantly.

20. The method of any one of claims 1-19, wherein the method decreases the patient's need for ventilator use.

21. The method of any one of claims 1-20, wherein the method reduces the incidence of death.

22. The method of any one of claims 1-21, wherein the method reduces the length of hospitalization for the patient or the number hospitalizations per number of patients.

23. The method of any one of claims 1-22, wherein the patient has not been hospitalized at the start of treatment.

24. The method of any one of claims 1-22, wherein the patient is already hospitalized at the start of treatment.

25. The method of claim 24, wherein the patient is in the ICU.

26. The method of any one of claims 1-25, wherein the patient has developed sepsis.

27. The method of any one of claims 1-26, wherein the patient has developed pneumonia.

28. The method of any one of claims 1-27, wherein the patient has started to show one or more symptoms of ARDS.

29. The method of claim 28, wherein the one or more symptoms is selected from severe shortness of breath, labored and unusually rapid breathing, low blood pressure and confusion and extreme tiredness.

30. The method of any one of claims 1-29, wherein the patient belongs to a population at high risk of complications after COVID-19 infection.

31. The method of any one of claims 1-30, wherein the patient also suffers from heart disease, hypertension, diabetes and/or obesity.

32. The method of claim 31, wherein th patient also suffers from hypertention.

33. The method of claim 32, wherein the patient is receiving treatment for hypertension with one or more anti-hypertensives.

34. The method of claim 33, wherein the one or more anti-hypertensives are each independently selected from an ARB inhibitor and an ACE inhibitor.

35. The method of claim 33 or 34, wherein the hypertension is well controlled by the treatment with the one or more anti-hypertensives.

36. The method of claim 33 or 34, wherein the hypertension is not well controlled despite the treatment with the one or more anti-hypertensives.

37. The method of any one of claims 1-36, wherein the patient is over 60 years old, over 70 years old or over 80 years old.

38. The method of claim 37, wherein the age of the patient is 65 or older, 70 or older or 80 or older.

39. The method of any one of claim 1-38, wherein the patient is not of Hispanic or Latino ethnicity.

40. The method of any one of claim 1-38, wherein the patient is of Hispanic or Latino ethnicity.

41. The method of claim 39 or 40, wherein the patient is non-white.

42. The method of any one of claims 1-38, wherein the patient is black or African American.

43. The method of any one of claims 1-38, wherein the patient is an American Indian or an Alaska native.

44. The method of any one of claims 1-38, wherein the patient is an Asian.

45. The method of any one of claims 1-38, wherein the patient is a Native Hawaiian or a Pacific Islander.

46. The method of any one of claims 1-45, wherein the method further comprises administering to the patient an additional therapeutic agent.

47. The method of claim 46, wherein the additional therapeutic agent is selected from a PDE5 inhibitor, a PDE9 inhibitor, NO, a NO donor, an anticoagulant and an antiviral.
